(19) United States
(12) Patent Application Publication
(10) Pub. No.: US 2024/0200990 A1
(43) Pub. Date: Jun. 20, 2024

(54) METHOD OF MEASURING MEASURANDS OF A MEDIUM AND A MEASUREMENT SYSTEM CONFIGURED TO PERFORM THIS METHOD

(71) Applicant: Endress+Hauser Conducta GmbH+Co. KG, Gerlingen (DE)

(72) Inventors: Julia Mildner, Gerlingen (DE); Rebecca Page, Basel (CH); Dimitri Vaissiere, Rixheim (FR); Pavo Vrdoljak, Nesselwang (DE)

(21) Appl. No.: 18/544,798

(22) Filed: Dec. 19, 2023

(30) Foreign Application Priority Data
Dec. 19, 2022 (DE) 10 2022 133 822.3

(51) Int. Cl. G01D 18/00; G01D 21/02

(52) U.S. Cl. CPC G01D 18/00; G01D 21/02; G01D 2218/10

(57) ABSTRACT

A method of measuring measurands of a medium with a set of sensors installed in proximity to each other at a measuring point is described. The method comprises: with each sensor determining measured values of one of the measurands and parameter values of a parameter, wherein the same parameter is measured by each of the sensors. Based on the measured values and the parameter values, parameter compensated measurement results of each measurand are determined and provided. Based on the parameter values determined by all sensors, at least one parameter property exhibited by the parameter at the measuring point is determined. The method also includes monitoring the parameter measurements for each parameter property and detecting an impairment of the parameter measurements when the parameter values provided by the sensors become non-compliant to a criterium defined for the parameter values based on the parameter property, and providing a corresponding monitoring result.

### CROSS-REFERENCE TO RELATED APPLICATION

The present application is related to and claims the priority benefit of German Patent Application No. 10 2022 133 822.3, filed on Dec. 19, 2022, the entire contents of which are incorporated herein by reference.

### TECHNICAL FIELD

The present disclosure concerns a method of measuring measurands of a medium with a set of at least two sensors and a measurement system configured to perform this method including the set of sensors.

### BACKGROUND

Measurement systems including sensors configured to measure measurands are employed in a large variety of different applications including industrial applications, as well as laboratory applications.

Measurement results of measurands measured by sensors installed in a specific application are often employed to monitor, to regulate and/or to control the measurands, an operation of a plant or facility, e.g. a production facility, and/or at least one step of a process, e.g. a production process, performed at the application. For example, in a chemical production process, concentrations of reactants used in the production process and/or the concentration of analytes contained in pre-products, intermediate products and/or educts produced by the process can be monitored and a sequence of process steps of the production process can be scheduled, regulated and/or controlled based on the measured values of the measurands. Further liquid analysis measurement systems measuring measurands, such as a pH-value, a concentration of free chlorine and/or a turbidity of a medium, are e.g. employed in swimming pools, as well as in drinking water supply networks and water purification plants to monitor, to regulate and/or to control the quality of the water.

Depending on the specific application, an efficiency and/or a productivity of a production process, a product quality of products produced, the safety of operation of facilities, industrial plants and/or laboratories and/or the quality of the medium may by depend on the measurement accuracy of the measured values of the measurands.

In many applications, the measurement accuracy of individual sensors is affected by at least one parameter, e.g. a temperature the sensor is exposed to. In this case, the measured values determined by the respective sensor exhibit a parameter-dependent measurement error. As a countermeasure, sensors are often equipped with a parameter sensing element measuring the parameter, e.g. a temperature sensing element, and a compensation of the parameter-dependent measurement error of the measured values of the measurand determined by the sensor is performed based on parameter values measured with the parameter sensing element. Compensating the parameter-dependent measurement error of the measured values increases the measurement accuracy of the thus attained compensated measurement result of the measurand. Including the parameter sensing element in the sensor provides the advantage, that the parameter is measured at the exact location where it affects the measurement accuracy of the sensor.

Unfortunately, there remains a risk, that the parameter measurements performed by the sensor may become impaired, e.g. due to a defect or a malfunctioning of the parameter sensing element. Compensating a parameter dependent measurement error based on erroneous parameter measurements will in most cases lead to a significant reduction of the measurement accuracy of the thus attained compensated measurement results.

### SUMMARY

It is an object of the present disclosure, to provide a method of measuring measurands of a medium and a measurement system performing that method, that enables for impairments of parameter measurements employed to compensate parameter dependent measurement errors of the measurements of the measurands to be detected at an early stage.

This object is achieved by a method of measuring measurands of a medium with a set of at least two sensors installed in proximity to each other at a measuring point, the method comprising the steps of:
with each sensor continuously or repeatedly determining and providing measured values of at least one of the measurands of the medium and parameter values of a parameter measured by the respective sensor, wherein the same parameter, in particular a temperature, is measured by each of the sensors,
based on the measured values and the parameter values determining and providing parameter compensated measurement results of each measurand,
based on the parameter values determined by all sensors determining at least one parameter property exhibited by the parameter at the measuring point, and
monitoring the parameter measurements performed by the sensors based on the parameter values determined by the sensors by for each parameter property detecting an impairment of the parameter measurements when the parameter values provided by the sensors become non-compliant to at least one criterium defined for the parameter values based on the respective parameter property, and
providing a corresponding monitoring result.

The present disclosure recognizes, that due to the proximity of the sensors to each other at the measuring point, the true parameter values of the parameter each of the individual sensors is exposed to can be expected to be approximately identical or at least to be closely related or correlated in a manner that is characteristic for the measuring point at the specific application where the sensors are installed. This enables for parameter properties of the parameter to be determined based on the multitude of parameter values determined by all individual sensors, which reflect the true behavior of the parameter at the measuring point accurately even when the individual measurements of the parameter performed by the sensors may each suffer from a limited measurement accuracy and/or local fluctuations of the parameter. Correspondingly the method provides the advantage, that impairments of the parameter measurements are detected based the parameter properties at an early stage.

Due to the influence of the parameter on the measurement accuracy of the measurements of the measurands, impairments of the parameter measurements detected by this method may also be indicative of a corresponding impairment of the parameter compensated measurement results of the measurands. Thus, the method provides the advantage, that potential impairments of the measurement results caused by erroneous parameter measurements do not remain unnoticed. This enables for appropriate countermeasures to be taken in due time to prevent potentially impaired measurement results from causing harm or damage. As an example, a further use of potentially impaired measurement results can be prevented as a safety measure.

According to a first embodiment:
monitoring the parameter measurements includes for at least one or each detected impairment identifying the sensor performing impaired parameter measurements that caused the detection of the respective impairment as an impaired sensor, and/or
providing the monitoring result includes for at least one or each detected impairment issuing a warning or an alarm, providing and/or indicating a general notification, that the parameter measurements are impaired, providing and/or indicating a notification, that the parameter measurements performed by the identified impaired sensor(s) are impaired, and/or indicating the identified impaired sensor(s), and/or
a replacement or repair of the identified impaired sensor(s) is scheduled to be performed and/or performed immediately or at a later point in time.

According to a second embodiment:
the at least one parameter property includes a first parameter property given by a parameter reference value continuously or repeatedly determined as or based on a median or an average of the parameter values simultaneously determined by all sensors, and
the method step of monitoring the parameter measurements includes for each sensor determining an impairment of the parameter measurements performed by the respective sensor when the parameter values determined by the respective sensor deviate from the corresponding parameter reference values by more than a predetermined tolerance.

According to a third embodiment:
the at least one parameter property includes a second parameter property given by a reference distribution exhibited by the values of the parameter at the locations of the sensors at the measuring point,
the second parameter property is determined based on training data including the parameter values provided by the sensors during a training time interval, and
during monitoring an impairment of the parameter measurements is detected when a vector or a time series of vectors formed by the parameter values simultaneously measured by all sensors occurs outside the reference distribution.

According to a refinement of the third embodiment:
the reference distribution is determined as or based on a cluster consisting of cluster points given by vectors formed by the parameter values simultaneously measured by all sensors at a multitude of different points in time during the training time interval, and/or
for at least one or each impairment detected based on the second parameter property at least one of the sensors performing impaired parameter measurements that cause the vector(s) to occur outside the reference distribution is identified as an impaired sensor based on a direction in which the vector(s) exceed the reference distribution.

According to a fourth embodiment:
the at least one parameter property includes third parameter properties given by reference correlations between the values of the parameter at the locations of the sensors,
the third parameter properties are determined based on training data including the parameter values provided by the sensors during a training time interval by determining the reference correlations as or based on the correlations between the parameter values measured by the sensors during the training time interval, and
based on the third parameter properties an impairment of the parameter measurements is detected when correlations between the parameter values measured by the sensors deviate from the corresponding reference correlations by more than a predetermined threshold.

According to a refinement of the fourth embodiment, at least one sensor performing impaired parameter measurements that cause the correlations between the parameter values measured by the sensors to deviate from the corresponding reference correlations by more than the predetermined threshold is identified as an impaired sensor based on correlations between the parameter values of individual sub-groups or pairs of the sensors and the corresponding reference correlations.

According to a fifth embodiment, the parameter-compensated measurement result of the measurand(s) are determined:
by for each measurand performing a compensation of a parameter-dependent measurement error of the measured values of the respective measurand based on parameter reference values determined as or based on a median or an average of the parameter values simultaneously determined by all sensors,
based on and/or in a manner accounting for the monitoring result,
by for each measurand, that is measured by one of the sensors, that has been identified as an impaired sensor performing impaired parameter measurements, determining the parameter-compensated measurement results based on the parameter reference values and by for each other measurand determining the parameter-compensated measurement results based on the parameter values provided by the sensor measuring the respective measurand,
by during time intervals during which no impairment of the parameter measurements is detected, determining the parameter-compensated measurement results of each measurand based on the parameter values provided by the sensor measuring the respective measurand and during time intervals during which an impairment of the parameter measurements is detected, determining the parameter-compensated measurement results of each measurand based on the parameter reference values, or
determining the parameter-compensated measurement result of each measurand that is measured by an impaired sensor based on the parameter values measured by at least one or each presently unimpaired sensor, in particular based on mean parameter values given by a mean or an average of the parameter values simultaneously determined by the unimpaired sensors, or based on extrapolated parameter values of the parameter at the location of the respective impaired sensor, wherein the extrapolated parameter values are determined based on the positions of the sensors and the parameter values measured by the unimpaired sensor(s), and wherein each presently impaired sensor is provided by or determined based on the monitoring result and each unimpaired sensor is given by one of the sensors that is presently not identified as an impaired sensor.

According to a sixth embodiment, the method further comprises for at least one group of interrelated measurands included in the measurands measured by the sensors performing a combined monitoring method based on interrelations between the measurands included in the respective group and interrelations between the parameter at the locations of the sensors measuring the measurands included in the group, in particular a combined monitoring method including the method steps of:
based on training data included in the measured values and the parameter values determined and provided by the sensor performing the method steps of:
determining parameter compensated measurement results of each measurand based on the measured values and the parameter values determined by the sensor measuring the respective measurand,
based on interrelations identified between the thus determined measurement results determining at least one group of interrelated measurands, and
for each group determining a characteristic interrelationship between the measurement results of the measurands included in the respective and a characteristic interrelationship between the parameter values determined by the sensors measuring the measurands included in the group, and
during measurement of the measurands performing the method steps of:
determining the measurement results of each measurand or at least of each measurand included in the group(s) of measurands based on the measured values and the parameter value determined by the sensor measuring the respective measurand, and
based on the thus determined measurement results of the measurands for each group performing the method steps of:
detecting an impairment of the measurement results of the measurands included in the respective group when the measurement results of the measurands included in this group become non-compliant to the characteristic interrelationship between the measurement results determined for the respective group, and
in case an impairment of the measurement results of the measurands included in the group is detected, performing at least one of the steps of:
detecting an impairment of the parameter measurements, when the parameter values determined by the sensors measuring the measurands included in the respective group are non-compliant to the characteristic interrelationship between the parameter values determined by the sensors measuring the measurands included in the respective group,
detecting a potential impairment of the measurands included in the group and/or their measurement, when the parameter values determined the sensors measuring the measurands included in the respective group are compliant to the characteristic interrelationship between the parameter values determined by the sensors measuring the measurands included in the group, and
providing and/or indicating at least one of:
the detected impairment of the measurement results,
the detected impairment of the parameter measurements or the detected potential impairment of the measurands included in the group and/or their measurement,
the group based on which the impairment of the measurement results has been detected, the measurands included in this group and/or the sensors measuring the measurands included in this group.

According to a refinement of the sixth embodiment, for each group:
the characteristic interrelationship between the measurement results of the measurands included in the respective includes a characteristic distribution of the measurement results, a characteristic pattern described by the measurement results and/or characteristic correlations between the measurement results of the measurands included in the respective group, and/or
the characteristic interrelationship between the parameter values determined by the sensors measuring the measurands included in the group includes a characteristic distribution of the parameter values, a characteristic pattern described by the parameter values, and/or characteristic correlations between the parameter values determined by the sensors measuring the measurands included in the respective group.

According to a seventh embodiment, the method further includes the method step of:
with a measurement device determining and providing parameter values of the parameter of the medium entering or exiting the measuring point,
based on training data including the parameter values of the parameter determined by the measurement device and the parameter values determined by the sensors determining reference correlations between the values of the parameter at the different locations of the sensors at the measuring point and the values of the parameter at the position of the measurement device,
based on the refence correlations detecting a correlation impairment when the correlations between the parameter values determined by the measurement device and the parameter values measured by the sensors deviate from the corresponding reference correlations by more than a predetermined threshold, and
performing at least one of: issuing a warning or an alarm when a correlation impairment is detected, indicating at least one or each detected correlation impairment and/or providing a notification informing about the detected correlation impairment.

The present disclosure is also embodied in a measurement system for measuring measurands of a medium at a measuring point configured to perform the method according to the present disclosure and comprising:
a set of two or more sensors installed or configured to be installed in proximity to each other at the same measuring point, wherein each sensor is configured to continuously or repeatedly determine and provide measured values of at least one of the measurands and parameter values of a parameter, wherein the same parameter, in particular a temperature, is measured by each one of the sensors, and
computing means configured to:
determine and to provide parameter compensated measurement results of the measurands based on the measured values and the parameter values determined by the sensors,
determine at least one parameter property exhibited by the parameter at the measuring point based on the parameter values measured by all sensors,
monitor the parameter measurements by for each parameter property detecting an impairment of the parameter measurements when the parameter values provided by the sensors become non-compliant to at least one criterium defined for parameter values based on the respective parameter property, and
provide a corresponding monitoring result.

According to a first embodiment of the measurement system:
the measurement system is a liquid analysis system,
the sensors include a chlorine sensor, a pH-sensor, an oxidation reduction potential sensor, a conductivity sensor, a spectral absorption sensor, and/or a sensor measuring a pressure, a density, a turbidity, a concentration of an analyte comprised in the medium, an oxygen content, an ammonium content, a phosphor content or at least one other measurand,
the sensors are installed or configured to be installed next to each other on a flow through cell conducting the medium,
the computing means is entirely or at least partially included in a transmitter, a superordinate unit, an edge device and/or in the cloud,
the computing means is configured to determine the compensated measurement results of the measurands based on and/or in a manner accounting for the monitoring result, and/or
the measurement system includes:
a transmitter, a superordinate unit and/or an edge device connected to and/or communicating with each sensor and/or configured to determine and/or to provide the measurement results and/or the monitoring result,
a least one interface providing the measurement results and/or at least parts of the monitoring result,
at least one display displaying the measurement results and/or at least parts of the monitoring result, and/or
at least one measurement device configured to continuously or repeatedly determine and to provide parameter values of the parameter of the medium entering or exiting the measuring point.

The present disclosure further comprises a computer program comprising instructions which, when the program is executed by a computer, cause the computer to carry out the method disclosed herein based on data given by or including the measured values of the measurands and the parameter values of the parameter provided by the sensors and their time of measurement, as well as a computer program product comprising this computer program and at least one computer readable medium, wherein at least the computer program is stored on the computer readable medium.

### BRIEF DESCRIPTION OF THE DRAWINGS

The present disclosure and further advantages are explained in more detail below based on the embodiments shown in the figures of the drawing, wherein:

FIG. 1 shows a flow chart of a method of measuring measurands of a medium;

FIG. 2 shows a measurement system configured to perform the method shown in FIG. 1;

FIG. 3 shows a measurement system configured to perform the method shown in FIG. 1, and

FIG. 4 shows a flow chart of a combined monitoring method.

### DETAILED DESCRIPTION

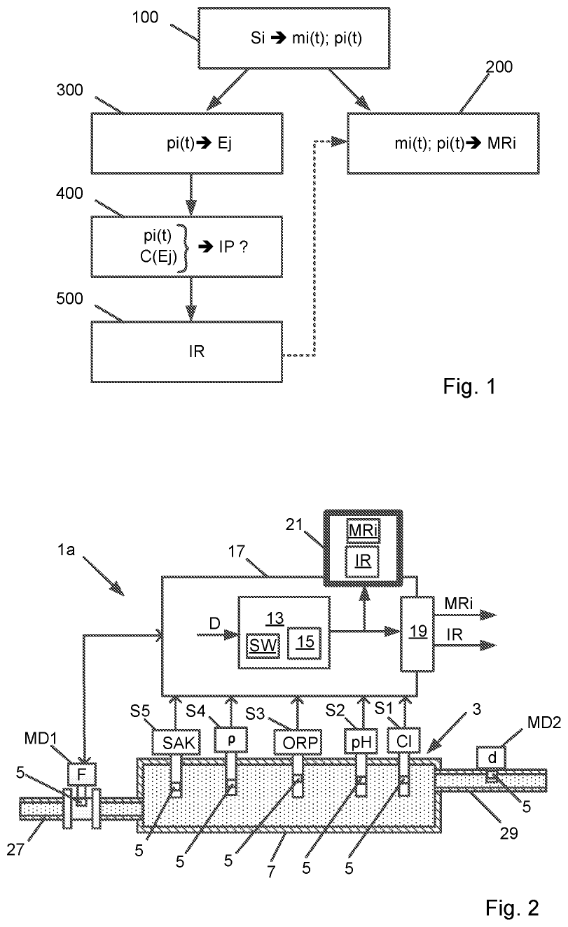

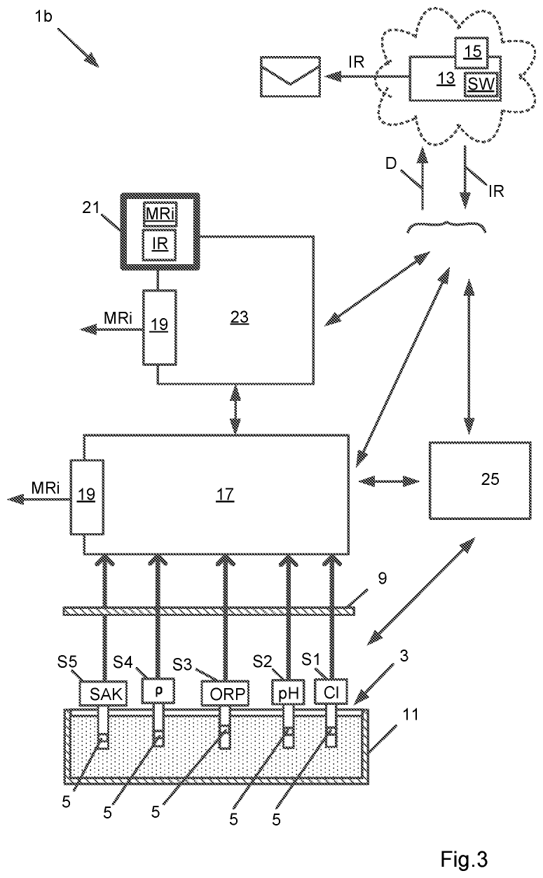

The present disclosure concerns a method of measuring measurands Mi of a medium, and a measurement system 1a, 1b configured to perform this method. A flow chart of the method is shown in FIG. 1. Examples of measurement systems 1a, 1b configured to perform this method are shown in FIGS. 2 and 3.

As shown, each measurement system 1a, 1b includes a set of two or more sensors Si installed or configured to be installed in proximity to each other at the same measuring point 3. Each sensor Si is configured to measure at least one of the measurands Mi and a parameter P affecting the measurement of the respective measurand Mi. The parameters P measured by each of the sensors Si are identical. Thus, the same parameter P is measured by each of the sensors Si at the location of the respective sensor Si. As an example, the parameter P measured by each of the sensors Si is e.g. a temperature the respective sensor Si is exposed to.

he method and/or the measuring systems 1a, 1b described herein can be applied in a multitude of different applications. Examples include industrial applications, e.g. production plants, chemical plants, water purification plants, as well as laboratory applications. Further examples include measurements in a natural environment, as well as measurement systems applied in medical diagnostics. In this respect, the sensors Si are e.g. each configured to measure at least one measurand Mi of interest at the specific application, e.g. a process parameter related to a process performed at the application and/or a property of the medium produced, processed and/or monitored at the application.

FIG. 2 shows an example, wherein the measurement system 1a is e.g. embodied as a liquid analysis system, e.g. a water quality measurement system for monitoring the quality of water in a specific application, e.g. at a drinking water supply network, where the sensors Si of the measurement system 1a are installed next to each other at the measurement point 3. The exemplary sensors Si shown in FIG. 2 include a chlorine sensor S1 measuring a concentration Cl of free chlorine, a pH-sensor S2 measuring a pH-value pH, an oxidation reduction potential sensor S3 measuring an oxidation reduction potential ORP, a conductivity sensor S4 measuring a conductivity p and/or a spectral absorption sensor S5 measuring a spectral absorption SAK of the medium. As shown, each sensor S1, S2, S3, S4, S5 includes a parameter sensing element 5 measuring the parameter P, e.g. a temperature sensing element measuring the temperature, of the medium. Depending on the application sets of sensors Si measuring other combinations of measurands Mi, e.g. measurands Mi including a pressure, a density, a turbidity, a concentration of an analyte comprised in the medium, an oxygen content, an ammonium content, a phosphor content and/or at least one other measurand Mi are e.g. employed.

Installation of the sensors Si in proximity to each other may be achieved in various ways. In FIG. 2 this is attained by installing the sensors Si next to each other on a flow through cell 7 conducting the medium. FIG. 3 shows an alternative embodiment, wherein the exemplary sensors S1, S2, S3, S4, S5 including the parameter sensing elements 5 shown in FIG. 2 are attached to a support 9 suspended above an open vessel 11, e.g. a tank, a container, or an open flow channel, such that the sensors Si extend into the medium contained in or flowing through the vessel 11 in proximity to each other.

As illustrated in FIG. 1, the method comprises a method step 100 of with each sensor Si continuously or repeatedly determining and providing measured values mi(t) of the measurand(s) Mi of the medium measured by the respective sensor Si and parameter values pi(t) of the parameter P.

The method further comprises a method step 200 of based on the measured values mi(t) and the parameter values pi(t) determining and providing parameter compensated measurement results MRi of each measurand Mi. To this extent, error compensation methods known in the art may be employed. Depending on the application where the method is used, the measurement results MRi of the measurands Mi provided by the measurement system 1a, 1b are e.g. employed to monitor, to regulate and/or to control a process performed at the application, e.g. on or by the plant or facility, to monitor, to regulate and/or to control at least one property or the quality of the medium, and/or to monitor, to regulate and/or to control an efficiency of a process performed at the application.

The method further comprises a method step 300 of based on the parameter values pi(t) provided by all sensors Si determining at least one parameter property Ej exhibited by the parameter P at the measuring point 3, a method step 400 of monitoring the parameter measurements performed by the sensors Si based on the at least one parameter property Ej, and a method step 500 of providing a corresponding monitoring result IR.

In method step 400 for each parameter property Ej an impairment IP of the parameter measurements performed by the sensors Si is detected, when the parameter values pi(t) provided by the sensors Si become non-compliant to at least one criterium C(Ej) defined for the parameter values pi(t) based on the respective parameter property Ej. In certain embodiments method step 400 may as an option include for at least one or each detected impairment IP identifying at least one of the sensors Si as an impaired sensor performing the impaired parameter measurements that caused the detection of the respective impairment IP.

The at least one parameter property Ej e.g. includes a first parameter property E1:=Pref(t) given by a parameter reference value Pref(t) continuously or repeatedly determined as or based on a median or average of the parameter values pi(t) simultaneously determined by the sensors Si. In these embodiments, the monitoring of the parameter measurements performed in method step 400 includes for each sensor Si detecting an impairment IP of the parameter measurements performed by the respective sensor Si when the parameter values pi(t) determined by the respective sensor Si deviate from the corresponding parameter reference values Pref (t) by more than a predetermined tolerance.

Monitoring the parameter measurements based on the first parameter property E1 provides the advantage, that the parameter reference values Pref(t) can be easily determined in real time, that it neither requires any knowledge about the application nor any preliminary training or learning to be performed, and that the impaired sensor(s) Si performing the impaired parameter measurements, that caused the detection of the impairment(s) IP are automatically identified. In this respect, the or each sensor Si determining parameter values pi(t) that deviate from the corresponding parameter reference values Pref (t) by more than the predetermined tolerance is identified as an impaired sensor.

In certain embodiments, the at least one parameter property Ej e.g. include a second parameter property E2 given by a reference distribution exhibited by the values of the parameter P at the locations of the sensors Si at the measuring point 3. The second parameter property E2 is preferably determined based on training data including the parameter values pi(t) provided by all sensors Si during a training time interval. The training time interval is a time interval during which all sensors Si are operating properly. In addition, the training time interval is preferably long enough, to cover all modes of operation that may occur at the measurement point 3 at the specific application, where the method is employed.

Based on the training data, the second parameter property E2 is then determined as or based on a distribution exhibited by the parameter values pi(t) measured by all sensors Si during the training time interval. As an example, the reference distribution is e.g. determined as or based on a cluster consisting of cluster points given by vectors formed by the parameter values pi(tn) simultaneously measured by all sensors Si at a multitude of different points in time tn during the training time interval.

Following the determination of the second parameter property E2, during monitoring an impairment IP of the parameter measurements is detected when a vector or a time series of vectors formed by the parameter values pi(ti) simultaneously determined by all sensors Si occurs outside the reference distribution. As an example, a vector is e.g. considered to be outside the reference distribution, when a statistical probability of the vector to constitute a sample of the reference distribution is smaller than a predetermined probability limit. As an option, in certain embodiments the or each impaired sensor Si performing impaired parameter measurements that cause the vector(s) to occur outside the reference distribution is e.g. identified as an impaired sensor based on the direction in which the vector(s) exceed the reference distribution.

In addition, or as an alternative, the at least one parameter property Ej e.g. includes third parameter properties E3 given by reference correlations between the values of the parameter P at the locations of the sensors Si at the measuring point 3. The third parameter properties E3 are preferably determined based on training data including the parameter values pi(t) determined by the sensors Si during a training time interval. To this extent, the training data described above in context with the second parameter property Ej is e.g. employed. Based on this training data, the reference correlations are e.g. determined as or based on the correlations between the parameter values pi(t) measured by the sensors Si during the training time interval. Subsequently, during monitoring an impairment IP of the parameter measurements is detected based on the third parameter properties E3 when the correlations between the parameter values pi(t) measured by the sensors Si deviate from the corresponding reference correlations by more than a predetermined threshold.

As an option, in certain embodiments, the or at least one sensor Si performing impaired parameter measurements that cause the correlations between the parameter values pi(t) measured by the sensors Si to deviate from the corresponding reference correlations by more than the predetermined threshold, is e.g. identified as an impaired sensor based on correlations between the parameter values pi(t) of individual sub-groups or pairs of the sensors Si and the corresponding reference correlations.

Based on the monitoring performed in method step 400, the method step 500 of providing the monitoring result IR e.g. includes for at least one or each detected impairment IP:
issuing a warning or an alarm,
providing and/or indicating a general notification, that the parameter measurements are impaired,
providing and/or indicating a notification, that the parameter measurements performed by the identified impaired sensor Si are impaired, and/or
indicating the identified impaired sensor(s) Si.

The method disclosed herein is preferably performed as a computer implemented method. In that case, at least the method steps 300, 400 and 500, preferably the method steps 200, 300, 400 and 500 are performed by computing means 13 executing a corresponding computer program SW based on data D given by or including the measured values mi(t) of the measurands Mi and the parameter values pi(t) of the parameter P provided by the sensors Si and their time of measurement t. Thus, the present disclosure is also realized in form of a computer program SW comprising instructions which, when the program is executed by a computer, cause the computer to carry out the method disclosed herein. In addition, the present disclosure further comprises a computer program product comprising this computer program SW and at least one computer readable medium, wherein at least the computer program SW is stored on the computer readable medium.

When the method is performed as a computer implemented method, the required data D is e.g. transferred to and at least temporarily stored in a memory 15 associated to the computing means 13. The computing means 13 or at least parts thereof are e.g. embodied as a unit including hardware, e.g. at least one microprocessor, a computer, or a computing system, located in the vicinity of the sensors Si.

In the embodiment shown in FIG. 2 the computing means 13 are included in a transmitter 17 of the measurement system 1a connected to or communicating with each sensor Si. In this embodiment, the transmitter 17 is e.g. is configured to determine and/or to provide the measurement results MRi and/or the monitoring results IR.

As shown in FIG. 3 the measurement system 1b may include a superordinate unit 23, e.g. a programmable logical controller, and/or an edge device 25 connected to or communicating with each sensor Si directly or via a transmitter 17 connected to or communicating with the sensors Si.

In this embodiment, the computing means 13 can at least in parts be included in the transmitter 17, the superordinate unit 23 and/or the edge device 25 configured to perform at least one of the method steps 200, 300, 400, 500 based on the measured values mi(t) and/or the parameter values pi(t) provided by the sensors Si. In that case, the transmitter 17, the superordinate unit 23 and/or the edge device 25 is e.g. is configured to determine and/or to provide the measurement results MRi and/or the monitoring results IR. FIG. 3 shows an alternative embodiment, wherein cloud computing is applied. Cloud computing denominates an approach, wherein IT-infrastructure, like hardware, computing power, memory, network capacity and/or software are provided via a network, e.g. via the internet. In that case, the computing means 13 is embodied in the cloud.

Depending on where the computing means 13 or parts thereof are located, the data D is directly or indirectly, e.g. via the transmitter 17, the superordinate unit 23 and/or the edge device 25 provided to the computing means 13 or the memory 15 associated to the computing means 13, e.g. as indicated by the double pointed arrows shown in FIG. 3. To this extent hard wired or wireless connections and/or communication protocols known in the art, like e.g. LAN, W-LAN, Fieldbus, Profibus, Hart, Bluetooth, Near Field Communication, TCP/IP etc. can be applied.

Regardless of whether the measurement results MRi and/or the monitoring results IR are entirely or at least in parts determined by the transmitter 17, the superordinate unit 23, the edge device 25 and/or in the cloud, the measurement results MRi and/or at least parts of the monitoring results IR are e.g. provided via at least one interface 19 of the measurement system 1a, 1b and/or displayed on at least one display 21 of the measurement system 1a, 1b. In FIG. 2, the interface 19 and the display 21 are included in or connected to the transmitter 17. In FIG. 3, the measurement results MRi are e.g. provided via the interface 19 included in or connected to the transmitter 17 determining or receiving the measurement results MRi and/or the interface 19 included in or connected to the superordinate unit 23 determining or receiving the measurement results MRi, and the measurement results MRi and/or the monitoring results IR are e.g. displayed by the display 21 included in or connected to the superordinate unit 23 determining or receiving the measurement results MRi and/or the monitoring results IR.

In addition, or as an alternative, as illustrated by the mail-symbol shown in FIG. 3, the entire monitoring result IR or at least parts thereof are e.g. provided in form of an e-mail or a message automatically generated by the computing means 13 and dispatched to a predetermined recipient and/or a predetermined device, e.g. a computer or a mobile device, e.g. a cell phone, a tablet, or a service tool.

In addition, or as an alternative, the entire monitoring result IR or at least parts thereof are e.g. provided to the transmitter 17 and/or the superordinate unit 23. This is particularly advantageous in embodiments of the method, wherein the measurement results MRi are determined based on the monitoring result IR and/or a manner accounting for monitoring result IR. In this case, the transmitter 17 or the superordinate unit 23 is e.g. configured to determine the measurement results MRi based on and/or in a manner accounting for monitoring result IR. As an additional or alternative option, the superordinate unit 23 is e.g. configured to regulate and/or to control a process performed at the application in a manner accounting for the monitoring result IR. As an example, the superordinate unit 23 is e.g. configured to perform at least one predefined action when the monitoring result IR fulfils a condition specified for the respective action. The predefined actions may include changing or stopping at least one process step of a process performed at the application. As an example, an entire process may be stopped by the superordinate unit 23 when an impairment IP is detected in an application, where potentially impaired measurement results MRi may have severe consequences regarding safety and/or costs.

The present disclosure provides the advantages mentioned above. Individual steps of the method and/or individual components of the measuring system 1a, 1b can be implemented in different ways without deviating from the scope of the present disclosure. Several optional embodiments are described in more detail below.

In this respect, different embodiments of the method step 200 of determining and providing the parameter-compensated measurement results MRi of the measurand(s) Mi can be employed.

As an example, the measurement results MRi are e.g. determined independently of the monitoring result IR. In this case the method and/or the measurement system 1a, 1b is e.g. configured to determine the parameter-compensated measurement results MRi of each measurand Mi by performing a compensation of a parameter-dependent measurement error of the measured values mi(t) of the respective measurand Mi based on the parameter values pi(t) measured by the sensor Si measuring the respective measurand Mi. In this embodiment, the measurement accuracy of the measurement results MRi of each measurand depends on the measurement accuracy of parameter values pi(t) measured by the sensor Si measuring the respective measurand Mi. Correspondingly, sensors Si that have been identified as impaired sensors performing impaired parameter measurements are preferably replaced or repaired immediately or at least as soon as possible after they have been identified.

As an alternative, the method and/or the measurement system 1a, 1b is e.g. configured to determine the parameter-compensated measurement results MRi of each measurand Mi by performing a compensation of a parameter-dependent measurement error of the measured values mi(t) of the respective measurand Mi based on the parameter reference values Pref (t) determined as or based on the mean or average of the parameter values pi(t) simultaneously determined by all sensors Si. This embodiment is particularly well suited for applications, wherein differences between the true value of the parameter P at the different locations of the sensors Si are negligible or at least smaller than a given threshold, e.g. a threshold determined as or based on a maximum permissible parameter measurement error.

Determining the parameter-compensated measurement results MRi based on the parameter reference values Pref (t) provides the advantage, that a reliable compensation of the parameter-dependent measurement error and a correspondingly high measurement accuracy of the measurement results MRi is ensured, even when the parameter measurements performed by one of the sensors Si are impaired or presently unavailable, e.g. due to a defect of the respective parameter sensing element 5. This provides the advantage, that a replacement or repair of each sensor Si, that has been identified as an impaired sensor performing impaired parameter measurements can safely be scheduled to be performed and/or performed at a later point in time, e.g. during a point in time, where it causes less or no disruptions of a process performed at the application, where the measurement system 1a, 1b is employed.

As another alternative, the parameter-compensated measurement result MRi of the measurand(s) Mi are e.g. determined based on the monitoring result IR and/or in a manner accounting for the monitoring result IR. This is indicated by the dotted arrow shown in FIG. 1.

In these embodiment, the method and/or the measurement system 1a, 1b is e.g. configured to determine the parameter-compensated measurement results MRi based on the parameter reference values Pref (t) only for those measurands Mi, that are measured by one of the sensors Si, that has been identified as an impaired sensor performing impaired parameter measurements, and based on the parameter value pi(t) provided by the sensor Si measuring the respective measurand Mi for all other measurands Mi.

As an alternative, the method and/or the measurement system 1a, 1b is e.g. configured to determine the parameter-compensated measurement results MRi of each measurand Mi based on the parameter values pi(t) determined by the sensor Si measuring the respective measurand Mi during time intervals during which no impairment IP of the parameter measurements is detected, and based on the parameter reference values Pref (t) during time intervals during which an impairment IP of the parameter measurements is detect.

As another alternative determining the parameter-compensated measurement result MRi of the measurand(s) Mi e.g. includes determining the parameter-compensated measurement result MRi of each measurand(s) Mi that is measured by one of the presently impaired sensors Si based on the parameter values pi(t) measured by at least one or each presently unimpaired sensor Si. In this embodiment, each presently impaired sensor Si is provided by or determined based on the monitoring result IR and each unimpaired sensor Si is given by one of the sensors Si that is presently not identified as an impaired sensor. Determining the parameter-compensated measurement result MRi of the measurand(s) Mi measured by the impaired sensor(s) based on the parameter values pi(t) measured by the unimpaired sensor(s) Si is e.g. performed by for each of these measurands Mi, performing a compensation of a parameter-dependent measurement error of the measured values mi(t) of the respective measurand Mi based on mean parameter values given by a mean or an average of the parameter values pi(t) simultaneously determined by the unimpaired sensors Si. As an alternative these compensations are e.g. performed based on extrapolated parameter values of the parameter P at the location of the impaired sensor Si determined based on the positions of the sensors Si and the parameter values pi(t) measured by the unimpaired sensor(s) Si.

Determining the measurement result MRi in a manner accounting for the monitoring result IR provides the advantage, that a reliable compensation of the parameter-dependent measurement error and a correspondingly high measurement accuracy of the measurement result MRi is ensured, even when the parameter measurements performed by one of the sensors Si are impaired or presently unavailable. Thus, replacement or repair of the identified sensor(s) Si can be or is e.g. scheduled to be performed and/or performed at a later point in time.

In certain embodiments, the method e.g. comprises for at least one group G of interrelated measurands Mi performing a combined monitoring method based on interrelation between the measurands Mi included in the respective group G and corresponding interrelations between the value of the parameter P at the locations of the sensors Si measuring the measurands Mi included in the group G.

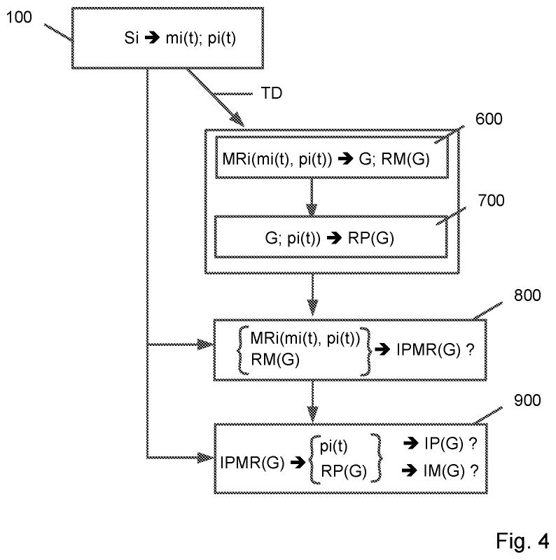

An example of this combined monitoring method is illustrated in the flow chart shown in FIG. 4. This combined monitoring method includes a method step 600 of based on training data TD included in the measured values mi(t) and the parameter values pi(t) determined and provided by the sensors Si in method step 100 determining parameter compensated measurement results MRi(mi(t), pi(t)) of each measurand Mi based on the measured values mi(t) and the parameter values pi(t) determined by the sensor Si measuring the respective measurand Mi. Based on these measurement results MRi(mi(t), pi(t)) at least one group G of interrelated measurands Mi is determined based on interrelations identified between the measurement results MRi(mi(t), pi(t)) of the measurands Mi. For each group G a characteristic interrelationship RM(G) between the measurement results MRi(mi(t), pi(t)) of the measurands Mi included in the respective G is determined. For each group G, the characteristic interrelationship RM(G) e.g. include(s) a characteristic distribution of the measurement results MRi(mi(t), pi(t)), a characteristic pattern described by the measurement results MRi(mi(t), pi(t)) and/or characteristic correlations between the measurement results MRi(mi(t), pi(t)) of the measurands Mi included in the respective group G.

The combined monitoring method further includes a method step 700 of based on the training data TD for each group G identified in method step 600, determining a characteristic interrelationship RP(G) between the parameter values pi(t) determined by the sensors Si measuring the measurands Mi included in the group G. For each group G, the characteristic interrelationship RP(G) between these parameter values pi(t) e.g. includes a characteristic distribution of the parameter values pi(t), a characteristic pattern described by the parameter values pi(t), and/or characteristic correlations between the parameter values pi(t) determined by the sensors Si measuring the measurands Mi included in the respective group G.

Following the preparatory steps 600 and 700, the combined monitoring method further includes a method step 800 of during measurement of the measurands Mi, determining the measurement results MRi(mi(t), pi(t)) of each measurand Mi or at least of each measurand Mi included in the group(s) G of measurands Mi based on the measured values mi(t) and the parameter value pi(t) determined by the sensor Si measuring the respective measurand Mi. Based on the thus determined measurement results MRi(mi(t), pi(t)) for each group G an impairment IPMR(G) of the measurement results MRi(mi(t), pi(t)) of the measurands Mi included in the respective group G is detected, when the measurement results MRi(mi(t), pi(t)) of the measurands Mi included in this group G become non-compliant to the characteristic interrelationship RM(G) between the measurement results MRi(mi(t), pi(t)) of the measurands Mi included in the respective G.

In case an impairment IPMR(G) of the measurement results MRi(mi(t), pi(t)) of the measurands Mi included in the group G or in one of the groups G is detected, the combined monitoring method further includes a method step 900 of determining whether the parameter values pi(t) determined by the sensors Si measuring the measurands Mi included in the respective group G are compliant or non-compliant to the characteristic interrelationship RP(G) between the parameter values pi(t) determined by the sensors Si measuring the measurands Mi included in the respective group G. Based on the outcome of this evaluation, an impairment IP(G) of the parameter measurements is detected, when the parameter values pi(t) determined by the sensors Si measuring the measurands Mi included in the respective group G are non-compliant to the characteristic interrelationship RP(G) between the parameter values pi(t) determined by the sensors Si measuring the measurands Mi included in the group G. In addition, or as an alternative, a potential impairment IM(G) of the measurands Mi included in the group G and/or their measurement is detected, when the parameter values pi(t) determined by the sensors Si measuring the measurands Mi included in the respective group G are compliant to the characteristic interrelationship RP(G) between the parameter values pi(t) determined by the sensors Si measuring the measurands Mi included in the group G.

In this embodiment providing the monitoring result IR e.g. includes providing and/or indicating:
at least one or each detected impairment IPMR(G) of the measurement results MRi(mi(t), pi(t)),
at least one or each detected impairment IP(G) of the parameter measurements, and/or
at least one or each detected potential impairment IM(G) of the measurands Mi included in the respective group G and/or their measurement.

In addition, or as an alternative, providing the monitoring result IR e.g. includes for each group G, based on which an impairment IPMR(G) of the measurement results MRi(mi(t), pi(t)) has been detected, providing and/or indicating the respective group G, the measurands Mi included in the respective group G and/or the sensors Si measuring the measurands Mi included in the respective group G.

In certain embodiments, the measurement system 1a may e.g. include at least one measurement device MD1, MD2 determining and providing parameter values pmd1(t), pmd2(t) of the parameter P of the medium entering or exiting the measuring point 3. This measurement device MD1, MD2 is e.g. parameter sensor measuring the parameter P. As an alternative, the measurement device MD1, MD2 is e.g. a device configured to measure a primary measurand and the parameter P. FIG. 2 shows an example, wherein the measurement device MD1 measuring the parameter P of the medium entering the flow cell 7 is a flow meter installed on a supply-pipe 27 connected to the flow cell 7 measuring the flow F of the medium entering the flow through cell 7, and wherein the measurement device MD2 measuring the parameter P of the medium exiting the flow cell 7 is a density sensor measuring a density d of the medium in a drain-pipe 29 connected to the flow cell 7. In FIG. 2 each measurement device MD1, MD2 includes a parameter sensing element 5 measuring the parameter P of the medium.

In these embodiments, the method e.g. includes for at least one of the measurement devices MD1, MD2 recording training data including the parameter values pmd1(t), pmd2(t) of the parameter P determined by the respective measurement device MD1 or MD2 and the parameter values pi(t) determined by the sensors Si. Next, based on this training data reference correlations between the values of the parameter P at the different locations of the sensors Si at the measuring point 3 and the values of the parameter P at the position of the respective measurement device MD1 or MD2 are determined.

Based on the thus determined reference correlations, during monitoring of the parameter measurements a correlation impairment is detected when the correlations between the parameter values pmd1(t), pmd2(t) determined by the respective measurement device MD1 or MD2 and the parameter values pi(t) measured by the sensors Si deviate from the previously determined reference correlations by more than a predetermined threshold.

In these embodiments providing the monitoring result IR e.g. includes issuing a warning or an alarm when a correlation impairment is detected, indicating at least one or each detected correlation impairment and/or providing a notification informing about the detected correlation impairment.

1. A method of measuring measurands of a medium with a set of at least two sensors installed in proximity to each other at a measuring point, the method comprising the steps of:
with each sensor continuously or repeatedly determining and providing measured values of at least one of the measurands of the medium and parameter values of a parameter measured by the respective sensor, wherein the same parameter is measured by each of the sensors;
based on the measured values and the parameter values determining and providing parameter compensated measurement results of each measurand;
based on the parameter values determined by all sensors determining at least one parameter property exhibited by the parameter at the measuring point;
monitoring the parameter measurements performed by the sensors based on the parameter values determined by the sensors by for each parameter property detecting an impairment of the parameter measurements when the parameter values provided by the sensors become non-compliant to at least one criterium defined for the parameter values based on the respective parameter property; and
providing a corresponding monitoring result.

2. The method according to claim 1, wherein:
monitoring the parameter measurements includes for at least one or each detected impairment identifying the sensor performing impaired parameter measurements that caused the detection of the respective impairment as an impaired sensor; and/or
providing the monitoring result includes for at least one or each detected impairment issuing a warning or an alarm, providing and/or indicating a general notification, that the parameter measurements are impaired, providing and/or indicating a notification, that the parameter measurements performed by the identified impaired sensor(s) are impaired, and/or indicating the identified impaired sensor(s); and/or
a replacement or repair of the identified impaired sensor(s) is scheduled to be performed and/or performed immediately or at a later point in time.

3. The method according to claim 1, wherein:
the at least one parameter property includes a first parameter property given by a parameter reference value continuously or repeatedly determined as or based on a median or an average of the parameter values simultaneously determined by all sensors; and
the method step of monitoring the parameter measurements includes for each sensor determining an impairment of the parameter measurements performed by the respective sensor when the parameter values determined by the respective sensor deviate from the corresponding parameter reference values by more than a predetermined tolerance.

4. The method according to claim 1, wherein:
the at least one parameter property includes a second parameter property given by a reference distribution exhibited by the values of the parameter at the locations of the sensors at the measuring point;
the second parameter property is determined based on training data including the parameter values provided by the sensors during a training time interval; and
during monitoring an impairment of the parameter measurements is detected when a vector or a time series of vectors formed by the parameter values simultaneously measured by all sensors occurs outside the reference distribution.

5. The method according to claim 4, wherein:
the reference distribution is determined as or based on a cluster consisting of cluster points given by vectors formed by the parameter values simultaneously measured by all sensors at a multitude of different points in time during the training time interval; and/or
for at least one or each impairment detected based on the second parameter property at least one of the sensors performing impaired parameter measurements that cause the vector(s) to occur outside the reference distribution is identified as an impaired sensor based on a direction in which the vector(s) exceed the reference distribution.

6. The method according to claim 4, wherein:
the at least one parameter property includes third parameter properties given by reference correlations between the values of the parameter at the locations of the sensors;
the third parameter properties are determined based on training data including the parameter values provided by the sensors during a training time interval by determining the reference correlations as or based on the correlations between the parameter values measured by the sensors during the training time interval; and
based on the third parameter properties an impairment of the parameter measurements is detected when correlations between the parameter values measured by the sensors deviate from the corresponding reference correlations by more than a predetermined threshold.

7. The method according to claim 6, wherein at least one sensor performing impaired parameter measurements that cause the correlations between the parameter values measured by the sensors to deviate from the corresponding reference correlations by more than the predetermined threshold is identified as an impaired sensor based on correlations between the parameter values of individual sub-groups or pairs of the sensors and the corresponding reference correlations.

8. The method according to claim 1, wherein the parameter-compensated measurement result of the measurand(s) are determined:
by for each measurand performing a compensation of a parameter-dependent measurement error of the measured values of the respective measurand based on parameter reference values determined as or based on a median or an average of the parameter values simultaneously determined by all sensors;
based on and/or in a manner accounting for the monitoring result;
by for each measurand, that is measured by one of the sensors, that has been identified as an impaired sensor performing impaired parameter measurements, determining the parameter-compensated measurement results based on the parameter reference values and by for each other measurand determining the parameter-compensated measurement results based on the parameter values provided by the sensor measuring the respective measurand;
by during time intervals during which no impairment of the parameter measurements is detected, determining the parameter-compensated measurement results of each measurand based on the parameter values provided by the sensor measuring the respective measurand and during time intervals during which an impairment of the parameter measurements is detected, determining the parameter-compensated measurement results of each measurand based on the parameter reference values; or
determining the parameter-compensated measurement result of each measurand that is measured by an impaired sensor based on the parameter values measured by at least one or each presently unimpaired sensor, in particular based on mean parameter values given by a mean or an average of the parameter values simultaneously determined by the unimpaired sensors, or based on extrapolated parameter values of the parameter at the location of the respective impaired sensor, wherein the extrapolated parameter values are determined based on the positions of the sensors and the parameter values measured by the unimpaired sensor(s), and wherein each presently impaired sensor is provided by or determined based on the monitoring result and each unimpaired sensor is given by one of the sensors that is presently not identified as an impaired sensor.

9. The method according to claim 1, further comprising for at least one group of interrelated measurands included in the measurands measured by the sensors performing a combined monitoring method based on interrelations between the measurands included in the respective group and interrelations between the parameter at the locations of the sensors measuring the measurands included in the group, in particular a combined monitoring method including the method steps of:
based on training data included in the measured values and the parameter values determined and provided by the sensor performing the method steps of:
determining parameter compensated measurement results of each measurand based on the measured values and the parameter values determined by the sensor measuring the respective measurand;
based on interrelations identified between the thus determined measurement results determining at least one group of interrelated measurands; and
for each group determining a characteristic interrelationship between the measurement results of the measurands included in the respective and a characteristic interrelationship between the parameter values determined by the sensors measuring the measurands included in the group; and
during measurement of the measurands performing the method steps of:
determining the measurement results of each measurand or at least of each measurand included in the group(s) of measurands based on the measured values and the parameter value determined by the sensor measuring the respective measurand; and
based on the thus determined measurement results of the measurands for each group performing the method steps of:
detecting an impairment of the measurement results of the measurands included in the respective group when the measurement results of the measurands included in this group become non-compliant to the characteristic interrelationship between the measurement results determined for the respective group; and
in case an impairment of the measurement results of the measurands included in the group is detected, performing at least one of the steps of:
detecting an impairment of the parameter measurements, when the parameter values determined by the sensors measuring the measurands included in the respective group are non-compliant to the characteristic interrelationship between the parameter values determined by the sensors measuring the measurands included in the respective group;
detecting a potential impairment of the measurands included in the group and/or their measurement, when the parameter values determined the sensors measuring the measurands included in the respective group are compliant to the characteristic interrelationship between the parameter values determined by the sensors measuring the measurands included in the group; and
providing and/or indicating at least one of:
the detected impairment of the measurement results;
the detected impairment of the parameter measurements or the detected potential impairment of the measurands included in the group and/or their measurement;
the group based on which the impairment of the measurement results has been detected, the measurands included in this group and/or the sensors measuring the measurands included in this group.

10. The method according to claim 9, wherein for each group:
the characteristic interrelationship between the measurement results of the measurands included in the respective includes a characteristic distribution of the measurement results, a characteristic pattern described by the measurement results and/or characteristic correlations between the measurement results of the measurands included in the respective group; and/or
the characteristic interrelationship between the parameter values determined by the sensors measuring the measurands included in the group includes a characteristic distribution of the parameter values, a characteristic pattern described by the parameter values, and/or characteristic correlations between the parameter values determined by the sensors measuring the measurands included in the respective group.

11. The method according to claim 1, further including the method step of:
with a measurement device determining and providing parameter values of the parameter of the medium entering or exiting the measuring point;
based on training data including the parameter values of the parameter determined by the measurement device and the parameter values determined by the sensors determining reference correlations between the values of the parameter at the different locations of the sensors at the measuring point and the values of the parameter at the position of the measurement device;
based on the refence correlations detecting a correlation impairment when the correlations between the parameter values determined by the measurement device and the parameter values measured by the sensors deviate from the corresponding reference correlations by more than a predetermined threshold; and
performing at least one of: issuing a warning or an alarm when a correlation impairment is detected, indicating at least one or each detected correlation impairment and/or providing a notification informing about the detected correlation impairment.

12. A measurement system for measuring measurands of a medium at a measuring point, comprising:
a set of two or more sensors installed or configured to be installed in proximity to each other at the same measuring point, wherein each sensor is configured to continuously or repeatedly determine and provide measured values of at least one of the measurands and parameter values of a parameter, wherein the same parameter, in particular a temperature, is measured by each one of the sensors, and
computing means configured to:
determine and to provide parameter compensated measurement results of the measurands based on the measured values and the parameter values determined by the sensors;
determine at least one parameter property exhibited by the parameter at the measuring point based on the parameter values measured by all sensors;
monitor the parameter measurements by for each parameter property detecting an impairment of the parameter measurements when the parameter values provided by the sensors become non-compliant to at least one criterium defined for parameter values based on the respective parameter property; and
provide a corresponding monitoring result.

13. The measurement system according to claim 12, wherein:
the measurement system is a liquid analysis system;
the sensors include a chlorine sensor, a pH-sensor, an oxidation reduction potential sensor, a conductivity sensor, a spectral absorption sensor, and/or a sensor measuring a pressure, a density, a turbidity, a concentration of an analyte comprised in the medium, an oxygen content, an ammonium content, a phosphor content or at least one other measurand;
the sensors are installed or configured to be installed next to each other on a flow through cell conducting the medium;
the computing means is entirely or at least partially included in a transmitter, a superordinate unit, an edge device and/or in the cloud;
the computing means is configured to determine the compensated measurement results of the measurands based on and/or in a manner accounting for the monitoring result, and/or
the measurement system includes:
a transmitter, a superordinate unit and/or an edge device connected to and/or communicating with each sensor and/or configured to determine and/or to provide the measurement results and/or the monitoring result;
a least one interface providing the measurement results and/or at least parts of the monitoring result;
at least one display displaying the measurement results and/or at least parts of the monitoring result; and/or
at least one measurement device configured to continuously or repeatedly determine and to provide parameter values of the parameter of the medium entering or exiting the measuring point.

14. A computer program comprising instructions which, when the program is executed by a computer, cause the computer to carry out a method, based on data given by or including the measured values of the measurands and the parameter values of the parameter provided by the sensors and their time of measurement, wherein the method includes:
with each sensor continuously or repeatedly determining and providing measured values of at least one of the measurands of the medium and parameter values of a parameter measured by the respective sensor, wherein the same parameter is measured by each of the sensors;
based on the measured values and the parameter values determining and providing parameter compensated measurement results of each measurand;
based on the parameter values determined by all sensors determining at least one parameter property exhibited by the parameter at the measuring point;
monitoring the parameter measurements performed by the sensors based on the parameter values determined by the sensors by for each parameter property detecting an impairment of the parameter measurements when the parameter values provided by the sensors become non-compliant to at least one criterium defined for the parameter values based on the respective parameter property; and
providing a corresponding monitoring result.

15. A computer program product comprising the computer program according to claim 14 and at least one computer readable medium, wherein at least the computer program is stored on the computer readable medium.
